(19) United States
(12) Patent Application Publication
(10) Pub. No.: US 2005/0191354 A1
(43) Pub. Date: Sep. 1, 2005

(54) Promoting cell regeneration and/or cell differentiation with non-metabolizable sugar and a polymeric absorbent

(76) Inventors: Daniel Jean, Vic Le Comte (FR); Leon Cariel, Paris (FR)

Correspondence Address: D Douglas Price;Steptoe & Johnson, 1330 Connecticut Avenue NW, Washington, DC, 20036, US

(21) Appl. No.: 10/466,619

(22) PCT Filed: Jan. 21, 2002
(86) PCT No.: PCT/FR02/00228

§ 371 Date: Feb. 11, 2004

(30) Foreign Application Priority Data
Jan. 19, 2001 (FR) 01/00738

(51) Int. Cl. A61K 31/715; A61K 9/14

(52) U.S. Cl. 424/486; 514/54

(57) ABSTRACT

A composition comprising at least one absorbent and at least one non-metabolizable sugar, and a method for using the composition, for promoting cell regeneration and/or cell differentiation.

The present invention relates to the use of a combination, comprising at least one absorbent and at least one compound selected from the group formed by nonmetabolizable sugars and polyols, for preparing a composition, in particular a medicinal composition, for promoting cellular reconstruction and/or cellular differentiation, in particular for promoting healing, advantageously the healing of bedsores.

The prior art discloses that it is possible to use sugars in the treatment of bedsores, in particular for fighting bacterial infections. The prior art is also found to point out that hydrophilic polymers can be used for treating bedsores with the aim of absorbing exudates.

The prior art which is presented below is extracted from the book “L'escarre-évaluation et prise en charge [The bedsore-assessment and care]”, which is coordinated by B. Barrois, D. Colin and S. Desjobert and published by Frison-Roche.

Bedsores are localized areas of tissue necrosis which are due to ischemia of the subcutaneous tissues. The main reason for the development of bedsores is a compression of the soft tissues between a bony protuberance and an external surface over an extended period of time.

In a general manner, the development of bedsores is explained by the prolonged pressure of said external surface on the skin and the underlying tissues and also by the phenomena of tissue hypoxia or anoxia which result from this.

Nevertheless, it has been observed that wide clinical heterogeneity exists and that, in certain patients, such as old people, persons with medullary injuries or comatose patients, it is not possible to regard the appearance of bedsores as being simply the result of the conjunction of a defect in care and an increase in tissue pressure. The appearance of localized bedsores in an area which is not a point of compression is frequently observed in certain patients, in particular patients in intensive care units.

Besides compression, other intrinsic factors are also known to play a role. These factors are, classically, shearing, maceration, infections and nutritional deficiency.

Shearing is mainly observed in patients who remain in a semi-sitting position for an extended period; it is engendered by the conjunction of hypertension and a tangential force which is linked to sliding.

Maceration is frequently the consequence of hyperthermia, which gives rise to increased transpiration and perspiration and which can be accompanied by sphincteral incontinence.

Infections also play a role, which can be all the more a determining factor since the patient may be subject to an immune deficiency.

Nutritional deficiency is manifested in tissue protein renewal, replenishment of the tissues with energy substrates and deficiency in vitamins or trace elements. The appearance of endogenous cachexia and, in particular, of diabetes can only aggravate this pre-existing situation.

Various products and techniques have been proposed for treating bedsores; the main ones are presented below.

Film dressings, which are thin, transparent, pliable and extendable sheets which are reserved for nonexudative bedsores.

Greasy dressings, which are made of tulle or gauze impregnated with vaseline, and which can additionally include an antibiotic, an antiseptic or a corticosteroid. The major drawback of these greasy dressings is that they only control the exudate poorly.

Hydrocolloids, on the contrary, absorb the exudate while gelling and maintain a humid environment. The problem posed by these products is that they disintegrate on contact with the exudates, producing a foul-smelling odor.

Alginates have great absorption power; they are useful when the wound is very exudative.

While hydrogels are less absorbent than alginates, their power of humidifying is very useful in the phase of cleaning the areas of necrosis, which phase precedes the healing phase. They suffer the disadvantage of being expensive.

Hydrocell dressings are very absorbent and control the exudate without abolishing it; their only indication is granulation.

The application of sugar to bedsores for the purpose of absorbing exudates has also been used as a treatment. However, this practice can suffer from drawbacks, in particular when the patient is diabetic, since, apart from the fact of increasing the blood sugar concentration in the patient, it has been found that, in these patients, healing is adversely affected. The consequence of supplying sugar to the wounds of these patients would be to aggravate this adverse effect on the healing process.

Surprisingly, it was possible to demonstrate, in in-vitro tests carried out on cell cultures, that the combination comprising at least one absorbent and at least one compound selected from the group formed by nonmetabolizable sugars and polyols very strongly promoted cellular differentiation and tissue reconstruction without exhibiting the abovementioned drawbacks.

The present invention therefore relates to the use of a combination, comprising at least one absorbent and at least one compound selected from the group formed by nonmetabolizable sugars and polyols, for preparing a composition, in particular a medicinal composition, for promoting cellular reconstruction and/or cellular differentiation.

This composition is intended, first and foremost, to be a medical tool, more especially a dressing item. Within the meaning of the present application, “nonmetabolizable sugar” is understood as being a sugar which cannot be used in its entirety as an energy source by the human body.

In particular, the present invention relates to the use of a combination, comprising at least one absorbent and at least one compound selected from the group formed by nonmetabolizable sugars and polyols, for preparing a composition, in particular a medicinal composition, for tissue reconstruction in human or veterinary medicine, examples of the application of which are as follows:
Injection, for making good wrinkles or deficits of dermal tissue or tissue underlying the skin, for the purpose of esthetic correction,
Reconstruction of nervous tissue: spinal cord trauma,
Bone reconstruction: deficit of bone tissue following interventions or traumas,
Reconstruction of cartilage: deficit associated with degenerative diseases or surgical interventions,
Treatment of burns,
Healing in general, advantageously prevention and treatment of bedsores.

The present invention also relates to the use of a combination, comprising at least one absorbent and at least one compound selected from the group formed by nonmetabolizable sugars and polyols, for preparing a composition, in particular a medicinal composition, for promoting cellular differentiation in medicine or in experimental biology:
Treatment of solid tumors,
Treatment of the bottom of cell culture dishes with the aim of promoting the attachment, differentiation, migration and growth of cells by replacing the matrices which are generally used for the same purpose but which are much more expensive in use and less versatile, such as collagen, fibronectin, polylysine or laminin or else various commercially available combinations of these substances.
Healing, including the treatment or prevention of bedsores.

The compound selected from the group formed by nonmetabolizable sugars and polyols is preferably selected from the group formed by xylose, arabinose, rhamnose, fucose, mannitol and sorbitol. Even more preferably, the compound will be xylose or arabinose, advantageously it will be xylose, which is commonly used in human diets as a substitute for sugar and which has demonstrated its innocuousness.

The absorbent is preferably a polymer; it can be selected, in particular, from polyacrylates, polymethacrylates, dextrans, alginates and carboxyvinyl polymer.

Advantageously, the polymer is an acrylic polymer.

In particular, it is selected from carboxyvinyl polymer (carbomer), sodium alginate, guar gum, polyacrylic acid, carboxymethyl cellulose, agar, agarose, xanthan gum, polyvinylpyrrolindone, methyl cellulose, poly(methyl methacrylate) and poly(acrylamide-co-acrylic acid). Advantageously, it is selected from carboxyvinyl polymer (carbomer), sodium alginate, guar gum, polyacrylic acid and carboxymethyl cellulose.

Even more advantageously, the polymer is carboxyvinyl polymer (carbomer).

Said composition is preferably present in the form of a powder. This formulation in the form of a powder thus exhibits the advantage of permitting an application which is easy and less painful than the applications of the prior art. Said composition in the form of a powder also exhibits the advantage of being strongly antiseptic by virtue of the combined effects of removal of the water which is required for bacterial survival, regular absorption of the exudates and the intense osmotic pressure which is achieved.

The compositions will preferably comprise between 50 and 95%, and even more preferably between 80 and 95%, by weight, of at least one compound selected from the group formed by nonmetabolizable sugars and polyols and between 5 and 50%, and even more preferably between 5 and 20%, by weight, of an absorbent.

This composition can also include an agent which is selected from antibiotics, antiseptics, corticosteroids, etc.

In addition, it has been established that the efficiency of the composition remains constant irrespective of whether the absorbent and the compound selected from the group formed by nonmetabolizable sugars and polyols are administered simultaneously, separately or such that the administrations are staggered over time.

The following examples are given by way of indication and are not limiting.

### EXAMPLE 1

### Demonstration of the Effect of the Combination of Carboxyvinyl Polymer (carbomer 914) and Xylose on the Migration, Attachment and Cellular Differentiation of Melanocytes Derived from a Continuous Cell Line

A 6-well culture dish is coated with an aqueous gel based on a mixture of carbomer 914 and xylose in the proportions 1-9 (w/w) to which distilled water is added so as to obtain a concentration of 1% (w/w). The gel which has been formed is neutralized to pH 7.00 with a N solution of sodium hydroxide and sterilized by autoclaving it at 120° C. for 20 minutes.

The resulting sterile gel is diluted 1/3 in a sterile manner with PBS buffer to which 34 μg of gentamycin have been added/ml. 1 ml of this diluted gel is placed in each well and the gel is allowed to dry overnight under a sterile laminar air flow.

Seeding is then carried out using a suspension containing 50 000 melanocyte cells from the M4Beu cell line (Tumourigenic phenotypes of human melanoma cell lines in nude mice determine by an active antitumor mechanism; R. Jacubovich, H. Cabrillat, D. Gerlier, M. Bailly & J. F. Doré; Br. J. Cancer (1985), 51, 335-345) in 3 ml of MEM culture medium containing 10% calf serum, 1% vitamin solution (ref. Sigma M 6895), 1% sodium pyruvate solution (ref. Sigma S 8636), 1% amino acid solution (ref. M 7145) and 50 μg of gentamycin/ml.

The cultures are placed in an incubator at 37° C. and 5% CO₂. Their growth is then examined.

Controls are carried out without coating the bottom of the wells.

Pronounced cellular differentiation is observed in the case of the carboxyvinyl polymer and xylose mixture, with this differentiation being associated with the formation of cell masses reminiscent of tissue formations.

Fairly good differentiation is also observed with sodium alginate and carboxymethyl cellulose on their own and in combination with xylose. Good results are also achieved when arabinose is used in place of the xylose.

These results, which are visible on the basis of cell morphology, are also evidenced by the synthesis of melanin by the melanocytes, with this synthesis representing a good marker of differentiation.

### EXAMPLE 2

### Demonstration of the Effect of the Combination of Polyacrylate and Xylose on the Treatment of Bedsores

Tests were carried out on 20 patients, 4 of whom presented with bedsores on the heel while the other 16 presented with sacral bedsores.

The bedsores on the heel measured from 1 to 3 cm in diameter at the beginning of the treatment. The sacral bedsores measured from 4 to 12 cm. In two of them, the sacrum was visible at the bottom of the lesion. All these bedsores were strongly exudative.

The treatment consisted in applying one composition in a powder form, comprising, by weight, 90% xylose and 10% polyacrylate, both morning and evening in such a way as to cover the whole of the interior surface of the lesion with an approximately 2 mm layer of powder.

A 75 to 100% reduction in the diameter of the bedsores, and complete disappearance of the pain, were observed in all cases without exception after one week of treatment.

The bedsores on the heel disappeared completely within ten days, merely leaving a discreet inflammation without any pain or itching.

The 11 least deep sacral bedsores all had a diameter of between 4 and 6 cm. These bedsores were almost entirely filled in within two weeks, leaving a simple depression which was discreetly inflammatory in the case of 3 of the bedsores or even without any inflammation in the case of the other 8 bedsores.

While the 5 most serious bedsores did not disappear entirely, epithelialization within the interior of the lesion was complete within two weeks. The final result presented as a loss of manner, which was very much less than the loss of material observed at the beginning of treatment, in the form of a depression which was more or less regular and more or less accentuated. The diameter of the lesion was observed to be reduced by from 75 to 85%.

1-19. (canceled)

20. A composition for promoting cellular reconstruction and/or cellular differentiation, comprising at least one absorbent and at least one nonmetabolizable sugar.

21. The composition as claimed in claim 20, wherein the composition is in an injectable form.

22. The composition as claimed in claim 20, wherein the nonmetabolizable sugar is xylose, arabinose, rhamnose or fucose.

23. The composition as claimed in claim 20, wherein the nonmetabolizable sugar is xylose or arabinose.

24. The composition as claimed in claim 20, wherein the nonmetabolizable sugar is xylose.

25. The composition as claimed in claim 20, wherein the absorbent is a polymer.

26. The composition as claimed in claim 20, wherein the absorbent is a polyacrylate, a polymethacrylate, a dextran, a carboxyvinyl polymer or an alginate.

27. The composition as claimed in claim 20, wherein the absorbent is sodium alginate, guar gum, polyacrylic acid, a carboxyvinyl polymer or carboxymethyl cellulose.

28. The composition as claimed in claim 20, wherein the absorbent is a carboxyvinyl polymer.

29. The composition as claimed in claim 20, wherein the composition is in the form of a powder.

30. The composition as claimed in claim 20, wherein the composition further comprises at least one antibiotic, antiseptic or corticosteroid.

31. The composition as claimed in claim 20, wherein the composition comprises between 50 and 95% by weight of the at least one nonmetabolizable sugar and between 5 and 50% by weight of the at least one absorbent.

32. The composition as claimed in claim 20, wherein the composition comprises between 80 and 95% by weight of the at least one nonmetabolizable sugar and between 5 and 20% by weight of the at least one absorbent.

33. A method for promoting cellular reconstruction and/or cellular differentiation, comprising administering the composition as claimed in claim 20 to an affected area in need of cellular reconstruction and/or cellular differentiation.

34. The method as claimed in claim 33, wherein the affected area is a wrinkle, deficit of dermal tissue or tissue underlying skin.

35. The method as claimed in claim 33, wherein the affected area is nerve tissue, bone, or cartilage in need of reconstruction.

36. The method as claimed in claim 33, wherein the affected area is a burn in need of healing.

37. The method as claimed in claim 33, wherein the affected area is a bedsore in need of alleviating or area susceptible to a bedsore.

38. The method as claimed in claim 33, wherein the affected area is a solid tumor.

39. The method as claimed in claim 33, wherein the affected area is a bottom of a cell culture dish to promote the attachment, differentiation, migration or growth of a cell.

40. The method as claimed in claim 33, wherein the absorbent and nonmetabolizable sugar are administered simultaneously, separately or staggered over time.
